(19) United States
(12) Patent Application Publication
(10) Pub. No.: US 2004/0170655 A1
(43) Pub. Date: Sep. 2, 2004

(54) Cosmetic and dermatological preparations comprising an isoflavone content and the use of isoflavones for producing cosmetic and dermatological preparations for reducing the sebum content of the skin

(76) Inventors: Stefan Gallinat, Wedel (DE); Kirsten Venzke, Hamburg (DE); Andreas Herpens, Reinbek (DE); Helga Biergiesser, Reinbek (DE); Franz Stab, Echem (DE)

Correspondence Address: GREENBLUM & BERNSTEIN, P.L.C., 1950 ROLAND CLARKE PLACE, RESTON, VA, 20191, US

(21) Appl. No.: 10/471,593

(22) PCT Filed: Mar. 21, 2002
(86) PCT No.: PCT/EP02/03162

(30) Foreign Application Priority Data
Mar. 23, 2001 (DE) 101 14 305.2

(51) Int. Cl. A61K 31/353; A61K 7/00

(52) U.S. Cl. 424/401; 514/456

(57) ABSTRACT

Use of isoflavones for producing preparations for reducing sebum production and/or for establishing a normal sebum homeostasis, as well as for the prophylaxis and treatment of greasy hair and dandruff.

The present invention relates to the use of topical preparations for reducing sebum production, for preserving normal sebum homeostasis, for removing sebum from the skin, in particular for removing and clearing comedones, for avoiding comedone formation for the prophylaxis and treatment of mild forms of acne, and for the prophylaxis and control of seborrhea and blemished skin.

Sebaceous gland overproduction and the possible skin disorders which develop as a result, such as sebum retention, formation of comedones in the region of the sebaceous glands, i.e. on the face (primarily forehead, nose and chin) and on the upper back, are commonly encountered skin problems which have hitherto not been solved to a satisfactory degree.

Sebum is the secretion of the sebaceous gland. Sebaceous glands are protrusions of the follicle epithelium and thus constituents of the follicle with which they form a functional unit. They are holocrine glands, i.e. the entire gland cell is for the greatest part converted into the secretion sebum. The cells of the sebaceous gland, which are continually renewed from below, become fat and decompose, and the skin sebum formed as a result is emptied onto the surface of the skin through the follicle openings.

According to G. Leonardy (J. Ft. Jellinek Cosmetology, Zweck und Aufbau kosmetischer Präparate [Purpose and structure of cosmetic preparations], Dr. Alfred Hüthig-Verlag, Heidelberg-Mainz-Basel, third completely revised and significantly expanded edition 1976, pages 26 to 29), skin sebum consists of mono-, di- and triglycerides (C₁₀-C₁₈), waxes (C₁₈-C₂₆), wax esters (C₂₈-C₃₈), normal saturated fatty acids (C₁₀-C₁₈), branched-chain saturated fatty acids (C₁₁-C₁₈), multibranched-chain saturated fatty acids (C₁₃-C₁₈), monounsaturated fatty acids (C₁₁-C₁₈), polyunsaturated fatty acids (C₁₃, C₁₅-C₁₇), sterols (cholesterol, 7-dehydrocholesterol, 7-hydroxycholesterol), branched and unbranched hydrocarbons (C₃₀-C₄₀), squalene and phospholipids.

Together with the aqueous secretion of the eccrine sweat glands, the lipids of the sebaceous gland form the hydro-lipid film of the skin. This surface film is an emulsion which may be a water-in-oil or an oil-in-water emulsion. It has the function of keeping the surface of the skin supple and of regulating the water content of the deeper layers of the skin. If the sebum is well hydrated, the water content is at least 10 to 20% by weight and the sebum is hydrophilic. If the hydrophilic-lypophilic balance of the surface film is disturbed and the water content decreases, then the sebum changes and becomes hydrophobic. The flow of sebum from the sebaceous gland and follicles is impeded. This results in the skin sebum being trapped in the follicle openings with this then, as a result, possibly leading to comedones and inflammations of the follicles.

The change in the skin sebum and the onset of comedone formation can have various causes. For example: external influences, such as incorrect cleansing habits and incorrect care, comodogenous substances in cosmetics, weathering influences, alkaline soaps and harsh detergents. Increased sebaceous gland secretion and the formation of comedones can also develop as a result of genetic factors and hormonal influences. Here too, comedones, inflammations, preacne and acne with its secondary effects may be the result.

The frequency of skin damage as a result of disturbed sebaceous gland function and disorders of the sebaceous glands is ever increasing, and the reversal/avoidance of: comedone formation is thus a pressing concern. However, attempts hitherto to solve comedone formation as a causal problem have led to results which are less than satisfactory.

As well as the manual removal of comedones by squeezing, numerous cleansing methods are known with which it is attempted to remove comedones and to permanently prevent comedone formation. These include special soaps, skin-peeling compositions and the like. Softening and astringent compositions are also used. Moreover, it is attempted to reduce the tendency for acne by adding drying, keratolytic, antiseborrheic and antibacterial active ingredients to cosmetic and pharmaceutical preparations, without irritations of the skin or drying of the skin arising.

However, skin cleansing degreases the skin and extracts moisture therefrom. In addition, soaps have the disadvantage that the water-insoluble calcium and magnesium salts of higher fatty acids, which form when the soaps are used in hard water, form slimy precipitates on the skin. Because they cannot be readily rinsed off, these precipitates remain for a relatively long period on the skin, block the follicle openings and can lead to the formation of comedones. For this reason, syndets (i.e. surfactants without soap character) are predominantly used in the form of washing creams or washing lotions for skin cleansing. Although these syndets do not form lime soaps, the treatment with highly surface-active agents has a greater degreasing and drying action than soap on the skin. The more often soap- and surfactant-containing products are applied to the skin, the more markedly their disadvantageous effects come to the fore, namely degreasing and drying of the skin as a result of destruction of the hydro-lipid film. The reduction in comedones almost always leads to a reduction in the water content in the upper layers of the skin and to a solid concrement formation in the sebaceous glands, which in turn can induce inflammations. However, the reduction in the content of moisture of the skin is counterproductive for a gentle removal of the comedones.

Seborrhea is an increased function of the sebaceous glands as a result of predisposition. Both scalp and skin on the face appear greasy. The composition of the seborrheic sebum is changed as compared with normal sebum. Three development stages of seborrhea are distinguished:

1. Simple seborrhea, mild cases, greasy after 8 days.

2. Oily seborrhea, greasy after just 2-3 days.

3. Irreversible form, can no longer be reversed. The seborrhea in which the hair appears to be bathed in grease after just one day.

The excessive secretion of the sebaceous glands can, inter alia, be triggered by androgenetic disorders and has a detrimental esthetic effect on the overall appearance of the hair. This disorder can also be the cause for alopecia which arises. A forerunner is in each case the seborrheic condition of the scalp. Vegetative disorders and inappropriate care can further worsen the appearance of the skin and also the condition of the hair. Even in cases of seborrhea, the hair may itself be dry as a result of disturbances in keratin formation. Dry, damaged hair is frequently caused by external stress such as, for example, sun or chemical treatments. Blow drying at too high a temperature or incorrect care of affected hair can lead to damage.

The causes of greasy hair lie within the human body and are hormonally determined. Each hair has its own sebaceous gland which produces grease (sebum). Sebum production is controlled hormonally, and over- or under-production may result, depending on the hormone sensitivity of the sebaceous gland. The sebum itself has the function of keeping the scalp supple. It passes from the sebaceous gland onto the scalp and only later to the hair root, where it is normally taken up by the hair shaft and remains invisible. In the case of the overproduction of sebum, the hair shaft is no longer able to accommodate this and the sebum becomes visible as a greasy film on the hair. The result is straggly, greasy-lustrous hair.

As a result of the fact that sebaceous gland production is dependent on the hormone balance, the problem of greasy hair cannot be solved fundamentally since the sebaceous glands continually produce grease. Consistent care and high-quality grooming routines continue to be the best method of controlling greasy hair.

Greasy hair has very troublesome consequences. The hair becomes straggly again just a short time after washing and the hairstyle does not hold.

Contrary to popular opinion, it is only a rumor that the hair becomes greasy more quickly as a result of excessively frequent washing. Mild shampoos for greasy hair ensure that excess grease is removed. Hair and scalp are supplied with sufficient moisture and counterbalance the overproduction of the sebaceous glands.

Greasy hair and dandruff are among the most common skin problems. These anomalies are to be attributed to a disturbance of sebaceous gland activity. If the sebaceous glands are hyperactive, the term used is seborrhea. In this connection, two forms can be differentiated: the oily form (seborrhea oleosa) and the dry form (seborrhea sicca).

Seborrhea Oleosa:

Here, the sebaceous glands are hyperactive, producing too much, and excessively oily, sebum. The skin therefore has a greasy shine, and the hair is greasy and straggly again just 2 to 3 days after washing, right into the tips.

Seborrhea Sicca:

This is likewise to be attributed to sebaceous glands being hyperactive, but the sebum is drier, and has a more solid consistency. With the small flakes of the epidermis, it forms large sebum flakes which are readily friable. The scalp has a wax-like shine, the hair becomes greasy only at the roots, the lengths and particularly the tips being dry and even brittle.

The treatment of seborrhea involves, firstly, regular and thorough washing of the head with special shampoos, which can be carried out as often as appears necessary. The washing should be combined with a massage in the connective tissue because this empties the sebaceous glands much more, which delays regreasing.

The object of the present invention is to provide a preparation which does not have the disadvantages of the known compositions used hitherto, which simultaneously has a sebum-dissolving, grease-regulating and care action, which removes comedones already present without irritation, which effectively prevents degreasing and drying of the skin, which brings about homeostasis of the sebum secretion to a normal state, which prevents comedone formation and the development of inflammatory skin phenomena, and improves existing skin inflammations and which equally cares for the skin.

This object is achieved according to the invention through the use of isoflavones for producing preparations for reducing the production of sebum.

Surprisingly, use of the preparation cancels the comedogenic effect of the raw materials used in the preparation, and thus the formation of comedones and, accordingly, the development of acne caused by cosmetic ingredients is prevented.

It has also been found that the active ingredients used according to the invention prevent the formation of seborrheic phenomena, in particular greasy hair, but also dandruff, and eliminate seborrheic phenomena which are already present, in particular greasy hair, but also dandruff.

Isoflavones are [lacuna] group belonging to the flavenoids, sometimes also referred to as isoflavenoids, of plant dyes, most of which are yellowish in color, which are derived from isoflavone. The unsubstituted basic body, the actual isoflavone (3-phenylchromone, 3-phenyl-4H-1-benzopyran-4-one) occurs, for example, in types of clover.

Some of the better known isoflavones are daidzein (4′,7-dihydroxyisoflavone), as 7-O-glucoside daidzin e.g. in soybean flour; genistein (4′,5,7-trihydroxyisoflavone) e.g. from soybeans and red clover; prunetin (4′,5-dihydroxy-7-methoxyisoflavone) e.g. from the bark of plum trees; blochanin A (5,7-dihydroxy-4′-methoxyisoflavone) e.g. from chick peas, red clover and other types of clover; orobol (3′,4′,5,7-tetrahydroxyisoflavone); santal (3′,4′,5-trihydroxy-7-methoxyisoflavone) e.g. from sandelwood, redwood and other woods; pratensein (3′,5,7-trihydroxy-4′-methoxyisoflavone) e.g. from fresh red clover or meadow clover.

Substitution schemes of a number of naturally occurring isoflavones are listed below:

Suitable isoflavones to be used according to the invention are, preferably, the isoflavones listed below:

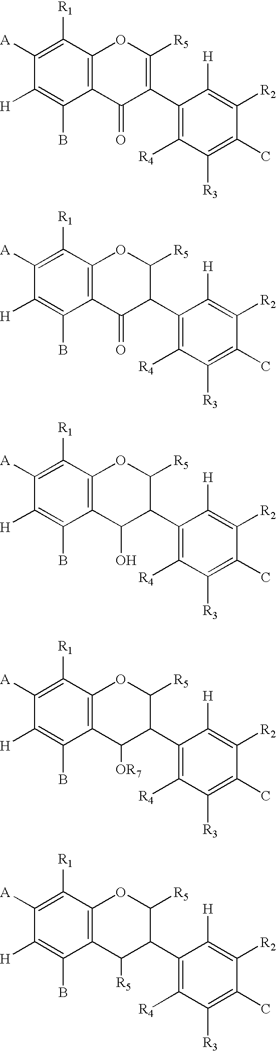

where A, B, C, R₁, R₂, R₃, R₄, R₅, R₇ and R₈ may be the following substituents: H, OH, OCH(CH₃)₂, OCH(C₂H₅)₂, OCH(C₃H₇)₂, OCH(CH₃)(C₂H₅), C₁-C₁₀-alkyl, O-glucopyranosyl, mono-, di-, oligosaccharides, acetyl, malonyl and OR₆ (where R₆=C₁-C₁₀-alkyl).

Of these, preference is in turn given to genistein, genistin, daidzein, daidzin, glycetein, glycetin, biochanin A, formononetin and ipriflavone, and derivatives of said isoflavones with the substituents described.

The invention thus also provides a method for the prophylaxis against and treatment of blemished skin, inflammatory skin conditions or seborrheic phenomena, in particular greasy hair and/or dandruff, characterized in that the active ingredients used according to the invention are brought, in a suitable cosmetic or dermatological carrier, into contact with the area affected by increased sebum production.

The prior art did not give the slightest indication to the inventive use as antiseborrheic active principle.

A further preferred embodiment of the present invention are thus formulations to be used against dandruff, for example antidandruff shampoos.

According to the invention, the active ingredients are preferably used in cosmetic or dermatological compositions [lacuna] a content of 0.0005-50.0% by weight, in particular 0.01-20.0% by weight, based on the total weight of the composition are preferred. Advantageously, the compositions comprise 0.02-10.0% by weight, particularly preferably 0.02-5.0% by weight, of the active ingredients used according to the invention, very particularly advantageously 0.5-3.0% by weight, in each case based on the total weight of the composition.

According to the invention, it may be advantageous to add further agents which are effective against sebum to the preparations according to the invention, for example chosen from the group consisting of distarch phosphate, cyclodextrin, sodium cornstarch octylene succinate, silica dimethyl silylate, hydroxypropyl distarch phosphates, tapiocadextrin, aluminum starch octylene succinate, acrylate copolymers, magnesium carbonate, cornstarch, aluminum starch octylene succinates, kaolin, nylon (e.g. nylon 6/12), bentonite, sodium silicoaluminate, boron nitride, silica, mica+titanium dioxide. In addition, it may be advantageous to encapsulate the active ingredients according to the invention, as solid lipid nanoparts using molten waxes, which may be chosen, inter alia, but not exclusively, from the group of ester waxes, triglyceride waxes or hydrocarbon waxes. In addition, it may be advantageous to encapsulate the active ingredient according to the invention in polymers, e.g. in particles based on highly crosslinked polymethacrylates and/or cellulose triacetates and/or as core/shell particles—particles with a shell made of poly(oxymethylurea), nylon, polyamides, polyurethane, polyesters, gelatins and polyolefins.

The combination of individual or two or more substances chosen from the above group with the isoflavones used according to the invention leads to a synergistic enhancement of the effect of the individual substances and thus is inventive in its own right.

The active ingredients used according to the invention can be incorporated without difficulties into customary cosmetic or dermatological formulations, advantageously into pump sprays, aerosol sprays, creams, ointments, tinctures, lotions, sticks, washing products and the like.

It is also possible and in some instances advantageous to combine the active ingredients used according to the invention with other active ingredients, for example with other antimicrobially, antimycotically or antivirally active substances.

It is advantageous to buffer the compositions according to the invention. A pH range from 3.0-9.0 is advantageous. It is particularly favorable to choose the pH in a range from 4.0-6.5.

The cosmetic and/or dermatological formulations according to the invention can have the customary composition and be used for the treatment of the skin and/or the hair in the sense of a dermatological treatment or a treatment in the sense of care cosmetics. They may, however, also be used in make-up products in decorative cosmetics.

For use, the cosmetic and/or dermatological formulations according to the invention can be applied in a sufficient amount to the skin and/or the hair in the manner customary for cosmetics and dermatological compositions.

According to the invention, customary antioxidants can be added to preparations which comprise the active ingredient combinations according to the invention.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene, phytoene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodiproplonate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. μmol to μmol/kg), and also (metal) chelating agents (e.g. α-hydroxy fatty acids, palmitic acid, phytic acid, lactoferrin), α-hydroxy acids (e.g. citric acid, lactic acid, malic acid), tetrahydrocurcumin, humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, alaninediacetic acid, flavonoids, polyphenols, catechins, vitamin C and derivatives (e.g. ascorbyl palmitate, Mg ascorbyl phosphate, ascorbyl acetate), tocopherols and derivatives (e.g. vitamin E acetate), and coniferyl benzoate of benzoin resin, rutinic acid and derivatives thereof, ferulic acid and derivatives thereof, butylhydroxytoluene, butylhydroxyanisole, nordihydroguaicic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these said active ingredients which are suitable according to the invention.

The amount of antioxidants (one or more compounds) in the preparations is preferably 0.001 to 30% by weight, particularly preferably 0.05-20% by weight, in particular 1-10% by weight, based on the total weight of the preparation.

The prophylaxis or the cosmetic or dermatological treatment with the active ingredient used according to the invention or with the cosmetic or topical dermatological preparations having an effective content of active ingredient used according to the invention is carried out in the usual manner, by applying the active ingredient used according to the invention or the cosmetic or topical dermatological preparations having an effective content of active ingredient used according to the invention to the affected areas of skin.

The active ingredient used according to the invention can advantageously be incorporated into customary cosmetic and dermatological preparations which may be in various forms.

Thus, they may, for example, be a solution, an emulsion of the water-in-oil (W/O) type or of the oil-in-water (O/W) type, or a multiple emulsion, for example of the water-in-oil-in-water (W/O/W) type or oil-in-water-in-oil (O/W/O) type, a hydrodispersion or lipodispersion, a gel, a solid stick or an aerosol.

Emulsions according to the invention for the purposes of the present invention, e.g. in the form of a cream, a lotion or a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient used according to the invention into aqueous systems or surfactant preparations for cleansing the skin and the hair.

The person skilled in the art is of course aware that demanding cosmetic compositions are mostly inconceivable without the customary auxiliaries and additives. Examples thereof include bodying agents, fillers, perfume, dyes, emulsifiers, additional active ingredients, such as vitamins or proteins, light protection agents, stabilizers, insect repellents, alcohol, water, salts, and antimicrobially, proteolytically or keratolytically active substances etc.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

In this connection, it is likewise advantageous to add the active ingredient used according to the invention as an additive to preparations which already comprise other active ingredients for other purposes.

It is, for example, advantageous for the purposes of the present invention to use a content of UV protection substances.

Preparations according to the invention can therefore advantageously comprise substances which absorb UV radiation in the UVB region, the total amount of filter substances being, for example, 0.1% by weight to 30% by weight, preferably 0.5 to 10% by weight, in particular 1 to 6% by weight, based on the total weight of the preparations.

The UVB filters can be oil-soluble or water-soluble. Examples of oil-soluble substances are:

3-benzylidenecamphor and derivatives thereof, e.g. 3-(4-methyl-benzylidene)camphor,

4-aminobenzoic acid derivatives, preferably 2-ethylhexyl 4-(dimethylamino)benzoate and amyl 4-(dimethylamino)benzoate;

esters of cinnamic acid, preferably 2-ethylhexyl 4-methoxycinnamate and isopentyl 4-methoxycinnamate;

esters of salicylic acid, preferably 2-ethylhexyl salicylate, 4-isopropylbenzyl salicylate and homomenthyl salicylate;

derivatives of benzophenone, preferably 2-hydroxy-4-methoxybenzophenone, 2-hydroxy-4-methoxy-4′-methylbenzophenone and 2,2′-dihydroxy-4-methoxybenzophenone;

esters of benzalmalonic acid, preferably di(2-ethylhexyl) 4-methoxybenzalmalonate;

2,4,6-trianilino(p-carbo-2′-ethyl-1′-hexyloxy)-1,3,5-triazine.

Advantageous water-soluble substances are:

2-phenylbenzimidazole-5-sulfonic acid and salts thereof, e.g. sodium, potassium or triethanolammonium salts,

sulfonic acid derivatives of benzophenones, preferably 2-hydroxy-4-methoxybenzophenone-5-sulfonic acid and the salts thereof;

sulfonic acid derivatives of 3-benzylidenecamphor, such as, for example, 4-(2-oxo-3-bornylidenemethyl)benzenesulfonic acid and 2-methyl-5-(2-oxo-3-bornylidenemethyl)sulfonic acid and the salts thereof.

The list of said UVB filters which can be used according to the invention is of course not intended to be limiting.

It can also be advantageous to use UVA filters which are customarily present in cosmetic and/or dermatological preparations in preparations according to the invention. Such filter substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)-propane-1,3-dione. Preparations which comprise these combinations are also provided by the invention. It is possible to use the same amounts of UVA filter substances as have been given for UVB filter substances.

Cosmetic and/or dermatological preparations for the purpose of the present invention can also comprise inorganic pigments which are customarily used in cosmetics for protecting the skin against UV rays. These are oxides of titanium, zinc, iron, zirconium, silicon, manganese, aluminum, cerium and mixtures thereof, and modifications in which the oxides are the active agents. Particular preference is given to pigments based on titanium dioxide. It is possible to use the amounts given for the above combinations.

The cosmetic and dermatological preparations used according to the invention can comprise cosmetic active ingredients, auxiliaries and/or additives as are customarily used in such preparations, e.g. antioxidants, preservatives, bactericides, perfumes, antifoams, dyes, pigments which have a coloring action, thickeners, surface-active substances, emulsifiers, emollients, moisturizers and/or humectants, fats, oils, waxes or other customary constituents of a cosmetic or dermatological formulation, such as alcohols, polyols, polymers, foam stabilizers, electrolytes, organic solvents or silicone derivatives.

If the cosmetic or dermatological preparation for the purposes of the present invention is a solution or emulsion or dispersion, solvents which may be used are:

water or aqueous solutions

oils, such as triglycerides of capric or caprylic acid, but preferably castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water can be a further constituent.

The oil phase of the emulsions, oleogels or hydrodispersions or lipodispersions for the purposes of the present invention is advantageously chosen from the group of esters formed from saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, and from the group of esters formed from aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24 carbon atoms, in particular 12-18 carbon atoms. The fatty acid triglycerides can, for example, be advantageously chosen from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂-₁₅-alkyl benzoate, caprylic/capric triglyceride, dicaprylyl ether.

Particularly advantageous mixtures are those of C₁₂-₁₅-alkyl benzoate and 2-ethylhexyl isostearate, those of C₁₂-₁₅-alkyl benzoate and isotridecyl isononanoate, and those of C₁₂-₁₅-alkyl benzoate, 2-ethylhexyl isostearate and isotridecyl isononanoate.

Of the hydrocarbons, paraffin oil, squalane and squalene are to be used advantageously for the purposes of the present invention.

Advantageously, the oil phase can also have a content of cyclic or linear silicone oils, or consist entirely of such oils, although it is preferred to use an additional content of other oil phase components apart from the silicone oil or the silicone oils.

Cyclomethicone (octamethylcyclotetrasiloxane) is advantageously used as the silicone oil to be used according to the invention. However, other silicone oils can also be used advantageously for the purposes of the present invention, for example hexamethylcyclotrisiloxane, polydimethylsiloxane and poly(methylphenylsiloxane).

Mixtures of cyclomethicone and isotridecyl isononanoate, and of cyclomethicone and 2-ethylhexyl isostearate are also particularly advantageous.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of—low carbon number, e.g. ethanol, isopropanol, 1,2-propanediol or glycerol and, in particular, one or more thickeners which can advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of Carbopols, for example Carbopol grades 980, 981, 1382, 2984 and 5984, in each case individually or in combination.

Gels used according to the invention usually comprise alcohols of low carbon number, e.g. ethanol, isopropanol, 1,2-propanediol, glycerol and water or an aforementioned oil in the presence of a thickener which, in the case of oily-alcoholic gels, is preferably silicon dioxide or an aluminum silicate, and, in the case of aqueous-alcoholic or alcoholic gels, is preferably a polyacrylate.

Solid sticks comprise, for example, natural or synthetic waxes, fatty alcohols or fatty acid esters.

Customary bases which are suitable for use as cosmetic sticks for the purposes of the present invention are liquid oils (e.g. paraffin oils, castor oil, isopropyl myristate), semisolid constituents (e.g. vaseline, lanolin), solid constituents (e.g. beeswax, ceresine and microcrystalline waxes and ozokerite) and high-melting waxes (e.g. carnauba wax and candelilla wax)

Suitable propellants for cosmetic and/or dermatological preparations which can be sprayed from aerosol containers for the purposes of the present invention are the customary known, readily volatile, liquefied propellants, for example hydrocarbons (propane, butane, isobutane), which can be used alone or in a mixture with one another. Compressed air is also used advantageously.

The person skilled in the art is of course aware that there are propellants which are nontoxic per se and are in principle suitable for realizing the present invention in the form of aerosol preparations, but which must nevertheless be avoided because of their unacceptable impact on the environment or other accompanying circumstances, in particular fluorocarbons and chlorofluorocarbons (CFCs).

For the purposes of the present invention, cosmetic preparations can also be in the form of gels which, in addition to an effective content of the active ingredient according to the invention and solvents customarily used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxypropylcellulose or hydroxypropylmethylcellulose, or inorganic thickeners, e.g. aluminium silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The examples below serve to illustrate the embodiments of the present invention. Unless stated otherwise, the data are always % by weight.

### 1. EXAMPLES OF O/W CREAMS

| | Examples | | | | | | | | | |
|---|---|---|---|---|---|---|---|---|---|---|
| | 1 | 2 | 3 | 4 | 5 | 6 | 7 | 8 | 9 | 10 |
| Glyceryl stearate citrate | 4.00 | | 2.00 | | 2.00 | | 2.00 | 2.00 | | |
| Glyceryl stearate SE | | 3.00 | | | | 5.00 | | | | |
| PEG-40 stearate | 1.00 | | | | | | | | | |
| Polyglyceryl-3 | | | | 3.00 | | | | | | |
| methylglucose distearate | | | | | | | | | | |
| Sorbitan stearate | | | | | 2.00 | | | | | |
| Stearic acid | | 1.00 | | | | | | | 2.50 | 3.50 |
| Ceteth-20 | | | | | | | | | | |
| Stearyl alcohol | | | 5.00 | | | 2.00 | | | | |
| Cetyl alcohol | 3.00 | 2.00 | | 3.00 | | | | | 3.00 | 4.50 |
| Cetylstearyl alcohol | | | | | 2.00 | | 3.00 | 1.00 | | 0.50 |
| C12-15 alkyl benzoates | | | | | | | 2.00 | 3.00 | | |
| Caprylic/capric triglyceride | 5.00 | 3.00 | 4.00 | 3.00 | 3.00 | 2.00 | | | | |
| Octyldodecanol | | | 2.00 | | 2.00 | 2.00 | 2.00 | | 4.00 | 6.00 |
| Dicapryl ether | | 4.00 | | 2.00 | 1.00 | | | | | |
| Mineral oil | 5.00 | 2.00 | | 3.00 | | | 4.00 | 2.00 | | |
| Cyclomethicone | | | | | | | | | 0.50 | 2.00 |
| Dimethicone | | | | | | 2.00 | | | | |
| TiO₂ | | | 1.00 | | | 2.00 | | | | |
| 4-Methylbenzylidenecamphor | | | 1.00 | | | 1.00 | | | | 1.00 |
| Butylmethoxydibenzoylmethane | | | 0.50 | | | 0.50 | | | | 0.50 |
| Glycine soya germ | 0.20 | 0.50 | 0.10 | 1.00 | 0.30 | 0.20 | 0.70 | 0.25 | 2.00 | 0.40 |
| extract (isoflavonoids) | | | | | | | | | | |
| Tococpherol | 0.1 | | | | 0.20 | | | | | 0.05 |
| Biotin | | | 0.05 | | | | | | | |
| Trisodium EDTA | 0.1 | | 0.10 | 0.1 | | | | 0.20 | | 0.20 |
| Preservative | q.s. | q.s. | q.s. | q.s. | q.s. | q.s. | q.s. | q.s. | q.s. | q.s. |
| Xanthan gum | | | | | | | | 0.20 | | |
| Carbomer | 3.00 | 0.1 | | 0.1 | 0.1 | 0.15 | 0.1 | | 0.05 | 0.05 |
| Sodium hydroxide solution 45% | q.s. | q.s. | q.s. | q.s. | q.s. | q.s. | q.s. | q.s. | q.s. | q.s. |
| Glycerol | 5.00 | 3.00 | 4.00 | 3.00 | 3.00 | 3.00 | | 3.00 | 5.00 | 3.00 |
| Butylene glycol | | 3.00 | | | | | 3.00 | | | |
| Alcohol, denat. | | | | | | | 3.00 | | 3.00 | |
| Perfume | q.s. | q.s. | q.s. | q.s. | q.s. | q.s. | q.s. | q.s. | q.s. | q.s. |
| Water | ad 100 | ad 100 | ad 100 | ad 100 | ad 100 | ad 100 | ad 100 | ad 100 | ad 100 | ad 100 |

### 2. EXAMPLES of W/O EMULSIONS

| | 1 | 2 | 3 | 4 | 5 | 6 | 7 |
|---|---|---|---|---|---|---|---|
| Cetyldimethicone copolyol | | 2.50 | | 4.00 | | | |
| Polyglyceryl-2 dipolyhydroxystearate | 5.00 | | | | 4.50 | 4.00 | 5.00 |
| PEG-30 dipolyhydroxystearate | | | 5.00 | | | | |
| Lanolin alcohol | | | | | | 0.50 | 1.50 |
| Isohexadecane | | | | | | 1.00 | 2.00 |
| Myristyl myristate | | | | | | 0.50 | 1.50 |
| Cera microcrystalline + | | | | | | 1.00 | 2.00 |
| Paraffinum liquidum | | | | | | | |
| Ethylhexyl methoxycinnamate | | 8.00 | | 5.00 | 4.00 | | |
| Anisotriazine | 2.00 | 2.50 | | 2.00 | 2.50 | | |
| Butylmethoxydibenzoylmethane | | | 2.00 | 1.00 | | 0.50 | 1.50 |
| Dioctylbutamidotriazone | 3.00 | 1.00 | | | 3.00 | | |
| Ethylhexyltriazone | | | 3.00 | 4.00 | | | |
| 4-Methylbenzylidenecamphor | | 2.00 | | 4.00 | 2.00 | 1.00 | 3.00 |
| Octocrylene | 7.00 | 2.50 | 4.00 | | 2.50 | | |
| Dioctylbutamidotriazone | 1.00 | | | 2.00 | | | |
| Bisimidazylate | 1.00 | 2.00 | 0.50 | | | | |
| Phenylbenzimidazolesulfonic acid | 0.50 | | | 3.00 | 2.00 | | |
| Titanium dioxide | | 2.00 | 1.50 | | 3.00 | | |
| Zinc oxide | 3.00 | 1.00 | 2.00 | 0.50 | | | |
| Mineral oil | | | 10.0 | | 8.00 | | |
| C12-15 alkylbenzoates | | | | 9.00 | | | |
| Dicaprylyl ether | 10.00 | | | | 7.00 | | |
| Butylene glycol | | | 2.00 | 8.00 | 4.00 | 4.00 | 5.00 |
| dicaprylate/dicaprate | | | | | | | |
| Dicaprylyl carbonate | 5.00 | | 6.00 | | | | |
| Dimethicone | | 4.00 | 1.00 | 5.00 | | | |
| Cyclomethicone | 2.00 | 25.00 | | | 2.00 | | |
| Shea butter | | | 3.00 | | | | 0.50 |
| PVP hexadecene copolymer | 0.50 | | | 0.50 | 1.00 | | |
| Butylene glycol | | | | | | | 6.00 |
| Octoxyglycerol | | 0.30 | 1.00 | | 0.50 | | 3.00 |
| Glycerol | 3.00 | 7.50 | | 7.50 | 2.50 | 5.00 | |
| Glycine soya | | 1.00 | 1.50 | | | | |
| MgSO₄ | 1.00 | 0.50 | | 0.50 | | | |
| MgCl₂ | | | 1.00 | | 0.70 | | |
| Vitamin E acetate | 0.50 | | 0.25 | | 1.00 | 0.50 | 1.00 |
| Glycine soya germ | 0.10 | 0.60 | 1.50 | 1.00 | 0.80 | 0.20 | 0.25 |
| extract (isoflavonoids) | | | | | | | |
| Trisodium EDTA | | | | | | 0.20 | 0.20 |
| DMDM hydantoin | | 0.60 | 0.40 | 0.20 | | | |
| Methylparaben | 0.50 | | 0.25 | 0.15 | | q.s. | q.s. |
| Phenoxyethanol | 0.50 | 0.40 | | 1.00 | 0.60 | q.s. | q.s. |
| Ethanol | 3.00 | | 1.50 | | 1.00 | | 3.00 |
| Perfume | q.s. | q.s. | q.s. | q.s. | q.s. | q.s. | q.s. |
| Water | ad 100 | ad 100 | ad 100 | ad 100 | ad 100 | ad 100 | ad 100 |

### 3. EXAMPLES OF HYDRODISPERSIONS

| | 1 | 2 | 3 | 4 | 5 |
|---|---|---|---|---|---|
| Ceteareth-20 | 1.00 | | | 0.5 | |
| Cetyl alcohol | | | 1.00 | | |
| Sodium carbomer | | 0.20 | | 0.30 | |
| Acrylates/C10-30-alkyl acrylate crosspolymer | 0.50 | | 0.40 | 0.10 | 0.10 |
| Xanthan gum | | 0.30 | 0.15 | | 0.50 |
| Ethylhexyl methoxycinnamate | | | | 5.00 | 8.00 |
| Anisotriazine | | 1.50 | | 2.00 | 2.50 |
| Butylmethoxydibenzoylmethane | 1.00 | | 2.00 | | |
| Dioctylbutamidotriazone | | 2.00 | | 2.00 | 1.00 |
| Ethylhexyltriazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Dioctylbutamidotriazone | 1.00 | | | 2.00 | |
| Bisimidazylate | 1.00 | | 0.50 | | 2.00 |
| Phenylbenzimidazolesulfonic acid | 0.50 | | | 3.00 | |
| Titanium dioxide | 0.50 | | 2.00 | 3.00 | 1.00 |
| Zinc Oxide | 0.50 | 1.00 | 3.00 | | 2.00 |
| C12-15-alkyl benzoates | 2.00 | 2.50 | | | |
| Dicaprylyl ether | | 4.00 | | | |
| Butylene glycol dicaprylate/dicaprate | 4.00 | | 2.00 | 6.00 | |
| Dicaprylyl carbonate | | 2.00 | 6.00 | | |
| Dimethicone | | 0.50 | 1.00 | | |
| Phenyltrimethicone | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Vitamin E acetate | 0.50 | | 0.25 | | 1.00 |
| Glycine soya germ extract (isoflavonoids) | 0.15 | 0.60 | 1.50 | 1.00 | 0.80 |
| DMDM hydantoin | | 0.60 | 0.40 | 0.20 | |
| Koncyl-L ® | q.s. | q.s. | q.s. | q.s. | q.s. |
| Methylparaben | q.s. | q.s. | q.s. | q.s. | q.s. |
| Phenoxyethanol | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

### 4. EXAMPLE (GEL CREAM):

| | Mass content (%) |
|---|---|
| Acrylates/C10-30 | 0.40 |
| alkyl-acrylatecrosspolymer | |
| Carbomer | 0.20 |
| Xanthan gum | 0.10 |
| Cetearyl alcohol | 3.00 |
| C12-15-alkyl benzoates | 4.00 |
| Caprylic/capric triglyceride | 3.00 |
| Cyclomethicone | 5.00 |
| Dimethicone | 1.00 |
| Glycine soya germ extract | 0.20 |
| (isoflavonoids) | |
| Glycerol | 3.00 |
| Sodium hydroxide | q.s. |
| Preservative | q.s. |
| Perfume | q.s. |
| Water, demineralized | ad 100.0 |

PH adjusted to 6.0

### EXAMPLE (W/O CREAM)

| | |
|---|---|
| Lameform TGI | 3.50 |
| Glycerol | 3.00 |
| Dehymuls PGPH | 3.50 |
| Glycine soya germ extract | 0.50 |
| (isoflavonoids) | |
| Preservative | q.s. |
| Perfume | q.s. |
| Magnesium sulfate | 0.6 |
| Isopropyl stearate | 2.0 |
| Caprylyl ether | 8.0 |
| Cetearyl isononanoate | 6.0 |
| Water, demin. | ad 100.0 |

### EXAMPLE (W/O/W CREAM)

| | Mass content (%) |
|---|---|
| Glyceryl stearate | 3.00 |
| PEG-100 stearate | 0.75 |
| Behenyl alcohol | 2.00 |
| Caprylic/capric triglyceride | 8.0 |
| Octylododecanol | 5.00 |
| C12-15-alkyl benzoates | 3.00 |
| Glycine soya germ extract | 1.00 |
| (isoflavonoids) | |
| Magnesium sulfate (MgSO₄) | 0.80 |
| EDTA | 0.10 |
| Preservative | q.s. |
| Perfume | q.s. |
| Water, demineralized | ad 100.0 |

PH adjusted to 6.0

1. The use of one or more isoflavones and derivatives thereof for producing preparations for reducing the production of sebum.

2. The use as claimed in claim 1 for preventing the formation and for the removal of comedones and/or for establishing normal sebum homeostasis.

3. The use as claimed in claim 1 for preventing or eliminating seborrheic phenomena, in particular for prophylaxis and treatment in the case of greasy hair, but also in the case of dandruff.

4. The use as claimed in claim 1, characterized in that the isoflavone or isoflavones are chosen from the group: 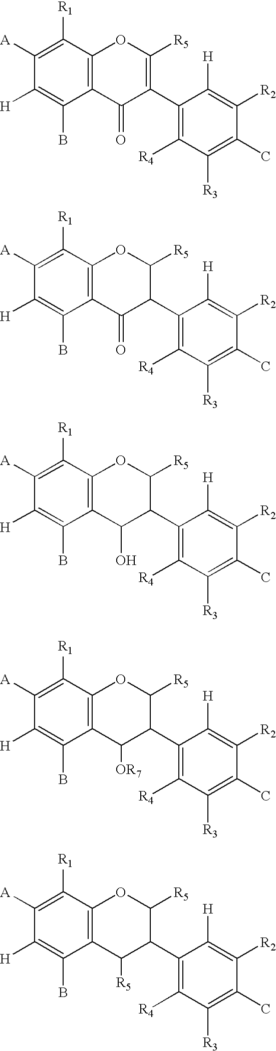
where A, B, C, R₁, R₂, R₃, R₄, R₅, R₇ and R₈ may be the following substituents: H, OH, OCH(CH₃)₂, OCH(C₂H₅)₂, OCH(C₃H₇)₂, OCH(CH₃)(C₂H₅), C₁-C₁₀-alkyl, O-glucopyranosyl, mono-, di-, oligosaccharide, amino acids, di- and oligopeptides, acetyl, malonyl and OR₆ (where R₆=C₁-C₁₀-alkyl).
Of these, preference is in turn given to genistein, genistin, daidzein, daidzin, glycetein, glycetin, biochanin A, formononetin and ipriflavone, and derivatives (see page 7-8).

5. A cosmetic method for the prophylaxis against and treatment of blemished skin or seborrheic phenomena, in particular greasy hair and/or dandruff, characterized in that the isoflavone or isoflavones are brought, in a suitable cosmetic or dermatological carrier, into contact with the area affected by increased sebum production.

6. The use as claimed in claim 1 or the method as claimed in claim 5, characterized in that the isoflavone or isoflavones are used corresponding to a content of 0.0005-50.0% by weight, in particular 0.01-20.0% by weight, based on the total weight of the composition.

7. The use as claimed in claim 1 or the method as claimed in claim 5, characterized in that the isoflavone or isoflavones are used corresponding to a content of 0.02-10.0% by weight, preferably 0.02-5.0% by weight, particularly advantageously 0.1-3.0% by weight, in each case based on the total weight of the composition. (See Gorbach!!)

8. A cosmetic or dermatological preparation with a content of one or more substances chosen from the group, where A, B, C, R₁, R₂, R₃, R₄, R₅, R₇ and R₈ may be the following substituents: H, OH, OCH(CH₃)₂, OCH(C₂H₅)₂, OCH(C₃H₇)₂, OCH(CH₃)(C₂H₅), C₁-C₁₀-alkyl, O-glucopyranosyl, mono-, di-, oligosaccharide, amino acids, di- and oligopeptides, acetyl, malonyl and OR₆ (where R₆=C₁-C₁₀-alkyl).
Of these, preference is in turn given to genistein, genistin, daidzein, daidzin, glycetein, glycetin, biochanin A, formononetin and ipriflavone.

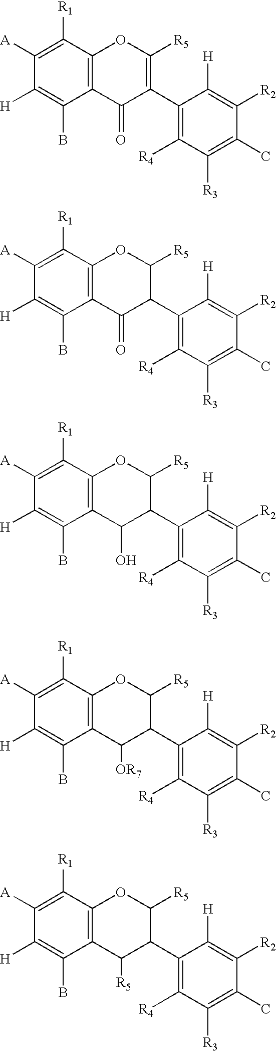

9. The preparation as claimed in claim 8, characterized in that the isoflavone or isoflavones are used corresponding to a content of 0.0005-50.0% by weight, in particular 0.01-20.0% by weight, based on the total weight of the composition.

10. The preparation as claimed in claim 8, characterized in that the isoflavone or isoflavones are used corresponding to a content of 0.02-10.0% by weight, preferably 0.02-5.0% by weight, particularly advantageously 0.1-3.0% by weight, in each case based on the total weight of the composition.
